(19) United States
(12) Patent Application Publication
(10) Pub. No.: US 2024/0344026 A1
(43) Pub. Date: Oct. 17, 2024

(54) Methods to Activate Gamma Delta T cells for Antibody Dependent Cellular Cytotoxicity

(71) Applicant: Intellistem, Inc., San Francisco, CA (US)

(72) Inventors: Anna BOLD, Neustadt/Aisch (DE); Timm HOERES, Reiskirchen (DE); Martin WILHELM, Nürnberg (DE)

(21) Appl. No.: 18/600,990

(22) Filed: Mar. 11, 2024

Related U.S. Application Data
(60) Provisional application No. 63/459,735, filed on Apr. 17, 2023.

(30) Foreign Application Priority Data
Feb. 26, 2024 (CA) 3230173

(51) Int. Cl. C12N 5/0783; A61P 35/00

(52) U.S. Cl. CPC C12N 5/0636; A61P 35/00; C12N 2500/32; C12N 2500/42; C12N 2501/2302; C12N 2501/2315; C12N 2501/998; C12N 2527/00

(57) ABSTRACT

A method for cultivating Gamma Delta T cells for immunotherapy, said method comprising: (i) obtaining peripheral blood mononuclear cells (MNCs) from a donor; (ii) optionally, isolating said MNCs by centrifugation; (iii) culturing said MNCs in a basal medium comprising fetal bovine serum, albumin and L-Glutamine; (iv) adding zoledronate to the medium; (v) adding a Interleukin-2 (IL-2) to the medium; and (vi) removing and replacing a portion of the medium with fresh medium.

### CROSS REFERENCE TO THE RELATED APPLICATIONS

This application claims the benefit of priority to Canadian Patent Application No. 3,230,173, filed Feb. 26, 2024, entitled “Methods to Activate Gamma Delta T cells for Antibody Dependent Cellular Cytotoxicity”, and claims the benefit of priority to U.S. Provisional Application No. 63/459,735, filed Apr. 17, 2023, entitled “Methods to Activate Gamma Delta T cells for Antibody Dependent Cellular Cytotoxicity”, the entirety of each is incorporated herein by reference.

### FIELD OF THE INVENTION

The present invention relates to T Cells and, in particular, to T Cells used in cellular immunotherapy and methods for culturing the same.

### BACKGROUND ART

Cellular immunotherapy is becoming increasingly important in the treatment of cancer. One of the newer approaches for immunotherapy involve the use of Gamma Delta T cells. In contrast to αβ T cells, the Gamma Delta T cell receptor (TCR) is not restricted to bind antigens in the context of a major histocompatibility complex (MHC) molecule. Part of the TCR are the variable Vγ and Vδ chains, with the Vγ 9Vδ 2 T cells being the dominant subpopulation and accounting for approximately 5% of T cells in peripheral blood. The anti-tumor effects of Gamma Delta T cells mediated by different mechanisms including cytokine production, perforin and interferon γ (IFN γ) release and antibody-dependent cell-mediated cytotoxicity (ADCC) have been demonstrated in detail in vitro and in vivo. Because of the MHC-independent recognition of target cells, no graft versus host reaction is to be expected with allogenic transfer of Gamma Delta T cells.

Strategies to use Gamma Delta T cells as cellular therapy include activation of either the patient's own Gamma Delta T cells or transferred allogeneic Gamma Delta T cells in vivo, or expansion and stimulation of autologous as well as allogeneic Gamma Delta T cells ex vivo with subsequent adoptive transfer. The expansion and stimulation of Vγ 9Vδ 2 T cells both in vivo and ex vivo is usually achieved via aminobisphosphonates or natural or synthetic phosphoantigens and addition of co-stimulators, mostly cytokines, like Interleukin 2 (IL-2). The aminobisphosphonate zoledronate (Zol) inhibits the farnesyl pyrophosphate synthase enzyme in the mevalonate pathway of antigen presenting cells like monocytes, which consequently leads to accumulation of isopentenyl pyrophosphate (IPP). IPP and its metabolites act as natural phosphoantigens bind to butyrophilin 3A1 (BNT3A1), which is detected by the TCR of Vγ 9Vδ 2 T cells with subsequent proliferation.

The number and functionality of patients' own Gamma Delta T cells is often limited (especially in cancer patients), so expansion and stimulation of these cells is usually unsuccessful both in vivo and ex vivo.

A disadvantage of in vivo activation is the toxic effect of IL-2 in higher concentrations and of zoledronate when used in short intervals. Additionally, the in vivo stimulability of Gamma Delta T cells decreases with repeated administrations of the activators.

Numerous cultivation methods have been published to stimulate Gamma Delta T cells ex vivo, but many of them require the use of a cell culture medium with fetal bovine serum (FBS). Using xenogeneic products bears the risk of contamination with known and even unknown pathogens and transmission of zoonotic diseases. In addition, such protocols often require culturing in plates and manual splitting, which is not compatible with a closed, sterile cultivation system advantageous for implementation of a GMP compliant process.

It would be advantageous to develop a method for cultivating Gamma Delta T cells to be used for immunotherapy, where the method addresses the above-mentioned issues either in part of in whole.

### SUMMARY OF THE INVENTION

In one aspect, the present invention provides a method for cultivating Gamma Delta T cells for immunotherapy, said method comprising: (i) obtaining peripheral blood mononuclear cells (MNCs) from a donor; (ii) optionally, isolating said MNCs by centrifugation; (iii) culturing said MNCs in a basal medium comprising fetal bovine serum, albumin and L-Glutamine; (iv) adding zoledronate to the medium; (v) adding a Interleukin-2 (IL-2) to the medium; and (vi) removing and replacing a portion of the medium with fresh medium.

In another aspect, the present invention provides Gamma Delta T cells for immunotherapy, said Gamma Delta T cells made by the method of the present invention.

### DESCRIPTION OF THE DRAWINGS

FIG. 1 illustrates proliferation, purity and anti-tumor efficiency of Gamma Delta T cells by using a preferred embodiment of the present invention for ex vivo stimulation;

FIG. 2 illustrates comparison of the cytotoxicity of γδ T cells after removal of αβ T cells and NKp46+ cells;

FIG. 3 illustrates indirect correlation of cell concentration and IFN production of stimulated Gamma Delta T cells;

FIG. 4 illustrates correlation of donor characteristics with the proliferation and anti-tumor activity of their stimulated Gamma Delta T cells;

FIG. 5 illustrates modification of proliferation and purity of Gamma Delta T cells by changing the stimulants of the activation protocol; and

FIG. 6 illustrates modification of anti-tumor activity and differentiation of Gamma Delta T cells by changing the stimulants of the activation protocol.

### DESCRIPTION OF PREFERRED EMBODIMENTS OF THE INVENTION

To establish a GMP-compliant and effective cultivation of Gamma Delta T cells, the present inventors developed a new protocol. In a preferred embodiment, the present inventors replaced the previous cultivation media, also known as “R10F”, which contains fetal calf serum, with the xeno-free OpTmizer™ CTS™ T cell expansion medium. In order to achieve an optimal result in terms of yield, the present inventors further increased the concentrations of zoledronate and IL-2 and did not administer the first IL-2 addition until day 2. Furthermore, the present inventors used flasks instead of well plates to facilitate upscalability. Assuming that the nutrient supply to the cells is limited by diffusion due to sedimentation in standing flasks, the present inventors placed them on an orbital shaker from day 4 of cultivation, but not earlier because of the need of the cell contact between monocytes and Gamma Delta T cells for stimulation.

Table 1 summarizes the relevant differences of the protocols of the prior art (also referred to as R10F) and a preferred embodiment of the present invention for ex vivo stimulation of Gamma Delta T cells;

TABLE 1

| | Prior Art | Present Invention |
|---|---|---|
| Media | RPMI 1640 Media | OpTmizer ™ T-Cell |
| | | Expansion SFM |
| Supplements | Fetal bovine serum, | Glutamine |
| | Glutamine, Penicillin/ | |
| | Streptomycin | |
| Culture plate | 96-well U-plate | 50 ml flask |
| | (200 μl/well) | (10 ml/flask) |
| Stimulants | 1 μM Zol d0 | 1 μM Zol d0 |
| | 100 U/ml IL-2 d0, d7, | 1000 U/ml IL-2 d0, |
| | d10, d14, d17 | d4, d7, d9, d11, d14, |
| | | d17 |
| Others | | shaker from d4 |

### Preferred Method of Culturing Gamma Delta T Cells

Cell cultures were performed with peripheral blood obtained from healthy adult donors. Peripheral blood mononuclear cells (MNC) were isolated by density gradient centrifugation with Biocoll (Biochrom, Darmstand, Germany/Bio&SELL, Feucht, Germany). MNC were incubated up to 17 days at 37° C. and 5% CO₂.

For cultivation, MNC at a concentration of 5.0E+05/ml were cultured in 50 ml cell culture flasks (Sarstedt, Nuembrecht, Germany) using medium consisting of OpTmizer™ CTSTM T-Cell Expansion Basal Medium, OpTmizer™ CTSTM T-Cell Expansion Supplement (Gibco/Thermo Fisher, Waltham, USA) and 1% 200 mM L-Glutamin. 10 μM Zoledronate were added on day 0 and 1000 U/ml IL-2 were added on day 2. On days 4, 7, 9, 11 and 14 half of the medium was removed and replaced by fresh medium containing 2× 1000 U/ml IL-2 for a final concentration of 1000 U/ml. Cells were not split at any time. From day 4 on cell culture flasks were shaken at 250 rpm. For pulsing zoledronate, 100 μM Zol were added on day 0. After 4 hours incubating at 37° C. and 5% CO2, MNC were harvested, washed twice and reseeded at a concentration of 5.0E+05/ml in cell culture flasks with fresh medium. The addition of IL-2 and/or IL-15 from day 2 and further cultivation was done as described above.

In another preferred embodiment, activation of the Gamma Delta T cells is carried out using one of the preferred cytokine mixes below:
(i) IL-2 and IL-15 were diluted in PBS/5% HSA and mixed the day prior to the treatment in a flask and left overnight incubated at 37° C. and 5% CO₂.
(ii) Zeldronate and IL-2 were diluted in PBS/5% HSA and mixed the day prior to the treatment in a flask and left overnight incubated at 37° C. and 5% CO₂.

Cell counts and cell viability were established using a hemocytometer and the trypan blue exclusion method. Cell count and proliferation rate of Gamma Delta T cells was calculated on the basis of the cell number of the MNC and the percentage of Gamma Delta T cells on the MNC determined by flow cytometry.

MNC stimulated with the preferred protocol of the present invention consist mainly of Gamma Delta T cells (>90%) and further purification was generally not necessary. MNCs stimulated with R10F and Ko-Op consist mainly of γδ T cells, but especially after stimulation with R10F (prior art), also of αβ T cells and NK cells. If the product had too high a concentration of Alpha Beta T cells and NK cells to compare activity of Gamma Delta T cells cultivated in prior art or present invention mediums, Alpha Beta T cells and NKp46+ were depleted from stimulated MNC after 10 days of cultivation using the MidiMACS system and anti-TCR Alpha Beta and anti-NKp46 MicroBeads (all from Miltenyi Biotec, Bergisch Gladbach, Germany) according to manufacturer's instructions except using HSA (CSL Behring GmbH, Marburg, Germany) instead of BSA as part of the washing buffer. In order to prove the success of depletion, the cells were stained before and after depletion with anti-TCR Gamma Delta-FITC, anti-CD16-PE, anti-CD3-ECD and anti-CD56-PC5 to diversify the different cell populations.

### Proliferation, Purity and Anti-Tumor Efficiency of Gamma Delta T Cells by Using Preferred Embodiment of Present Invention for Ex Vivo Stimulation

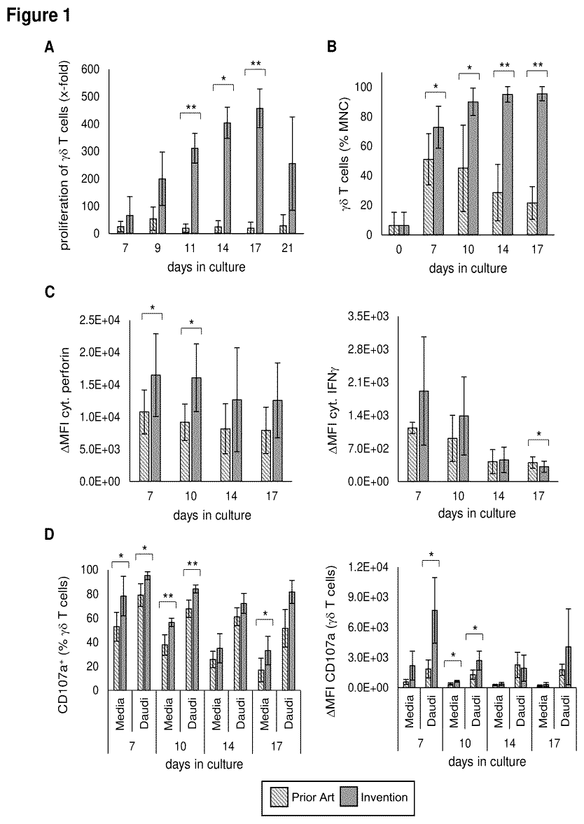

MNC of three healthy donors were isolated and stimulated with Zol/IL-2 using the protocols R10F (prior art) and a preferred embodiment of the present invention (also referred to as Ko-Op) over 21 days to compare the proliferation rate every two to four days. In terms of Gamma Delta T cells, a proliferation rate of more than 400-fold of the baseline can be achieved with the preferred embodiment of the present invention (see FIG. 1A).

For further testing, MNC were again isolated from healthy donors and cultivated with R10F (prior art) and a preferred embodiment of the present invention over 17 days. The percentage of Gamma Delta T cells determined by flow cytometry was significantly higher when the present invention was used for cultivation (see FIG. 1B).

To evaluate the activity of stimulated Gamma Delta T cells, cytoplasmic perforin and IFN γ were stained every three to four days and Δ MFI was measured by flow cytometry. Gamma Delta T cells produced significantly more perforin when cultured according to the preferred embodiment of the present invention, as well as more IFN γ after 7 and 10 days but not after 14 and 17 days (see FIG. 1C).

For investigation of the anti-tumor efficiency of stimulated Gamma Delta T cells, degranulation of Gamma Delta T cells after 3 h incubation with the tumor cell line Daudi or media only was determined by measuring the surface molecule CD107a by flow cytometry from day 7 onwards every three to four days. With and without tumor cells, stimulation with the preferred embodiment of the present invention increases both the percentage of CD107a+ Gamma Delta T cells and the amount of CD107a on the surface of Gamma Delta T cells as determined by Δ MFI compared to stimulation with prior art R10F (see FIG. 1D).

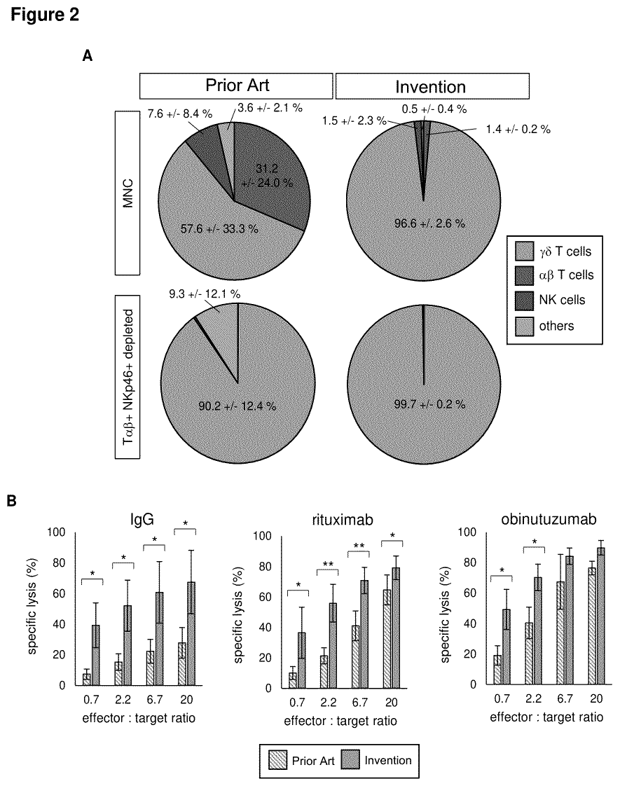

To directly determine the anti-tumor activity of Gamma Delta T cells against the tumor cell line Daudi, the present inventors performed a cytotoxicity assay on day 10 of cultivation comparing the prior art (R10F) and preferred embodiment of the present invention (Ko-Op). For this, Alpha Beta T cells and NKp46+ cells were paramagnetically depleted from the cell suspension in order to isolate the Gamma Delta T cells on day 10. The cell composition before and after depletion is shown FIG. 2A.

By cultivation using the preferred embodiment of the present invention compared to prior art (R10F), a significant increase in the cytotoxicity of Gamma Delta T cells against Daudi is achieved with both the monoclonal antibodies rituximab and obinutuzumab and their non-specific isotype control (see FIG. 2B).

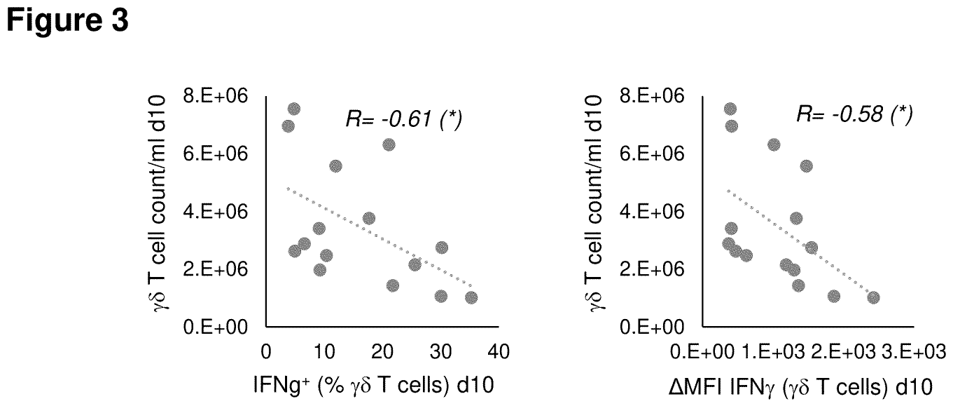

The present inventors tested the proliferation rate of MNC, the percentage of Gamma Delta T cells, the CD107a expression and the cytoplasmic IFN γ on day 10 for correlations. While, as expected, the proliferation rate and purity as well as IFN γ production and CD107a expression are significantly related (data not shown), there is a negative correlation between proliferation rate and IFN γ production (see FIG. 3).

In summary, cultivation with the preferred embodiment of the present invention enhances the proliferation rate, the purity, the anti-tumor activity and the cytotoxicity of stimulated Gamma Delta T cells over prior art.

### Correlation of Donor Characteristics with the Proliferation and Anti-Tumor Activity of Their Stimulated Gamma Delta T Cells

If several donors are available, it would be helpful being able to deduce from characteristics of the donors and their unstimulated Gamma Delta T cells whether their stimulated Gamma Delta T cells have the potential of high yield and exhibit high anti-tumor activity.

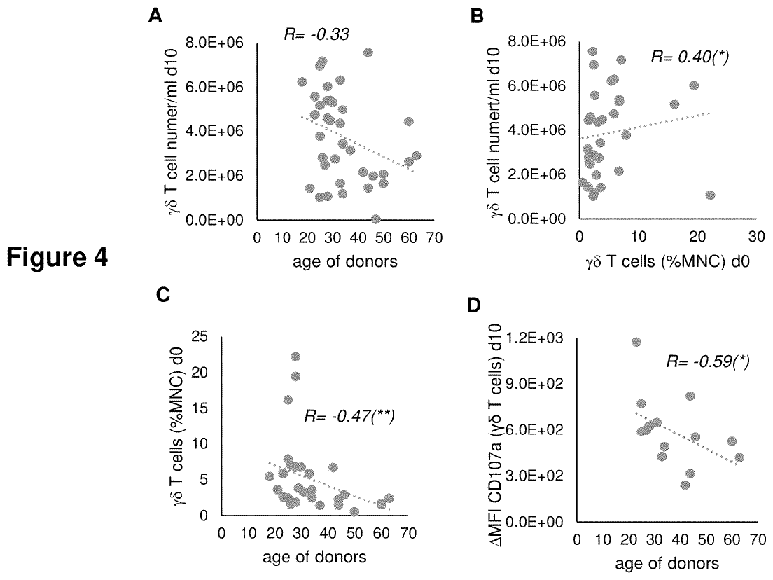

The present inventors could not find a significant correlation between the donors' sex and the cell concentration of Gamma Delta T cells on day 10 of stimulation (data not shown). Since the present inventors did not split cells during the cultivation with the present invention, the cell concentration is a direct indicator of cell count of Gamma Delta T cells. There is also no significant correlation between cell concentration at day 10 and the donors' age, although the significance level for the negative correlation between age and cell concentration of Gamma Delta T cells is just not reached with a p value of 0.052 (see FIG. 4A).

However, the present inventors observed a positive correlation between the cell concentration of Gamma Delta T cells on day 10 of stimulation and the percentage of Gamma Delta T cells of unstimulated MNC, which in turn correlates negatively with age (see FIGS. 4B, 4C).

Additionally, the present inventors found a negative correlation between age of donors and the ΔMFI of CD107a in the degranulation assay without incubation with Daudi cells (see FIG. 4D).

In conclusion, higher age seems to be associated with lower percentage of Gamma Delta T cells in MNC and therefore, with lower proliferation when stimulated. Additionally, the stimulated Gamma Delta T cells of older donors probably bear less anti-tumor activity compared to those of younger donors. Thus, the percentage of Gamma Delta T cells as well as the age of the donors can be used as a decision-making aid in the selection of donors.

### Modification of Proliferation, Purity and Anti-Tumor Activity of Gamma Delta T Cells by Changing the Stimulants

The present inventors next investigated whether there could be enhancement by a zoledronate pulse or by applying IL-15 in addition to or in place of IL-2, or by the combination of both. For this, MNC of healthy donors were isolated and stimulated according to different alterations concerning the zoledronate addition and the composition of the interleukins up to ten days. For zoledronate pulse, 100 μM zoledronate was added to the isolated MNC and washed out again after 4 hours of incubation. When IL-15 was used, this was at a concentration of 10 ng/ml, with the concentration of IL-2 remaining unchanged at 1000 U/ml.

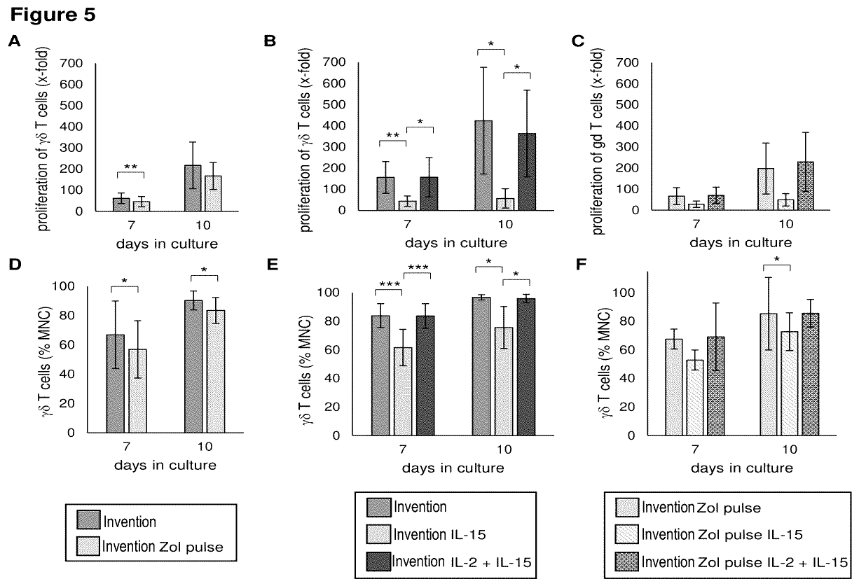

Pulsing zoledronate led to a significant lower proliferation rate of Gamma Delta T cells on day 7 of cultivation (see FIG. 5A).

The combined application of IL-2 and IL-15 led to no significant change in proliferation rate of Gamma Delta T cells, and the proliferation was significantly lower when II-15 was used alone (see FIG. 5B).

The combination of pulsing the zoledronate and adding IL-2 and IL-15 also did not lead to any significant improvement (see FIG. 5C).

The proportion of Gamma Delta T cells and thus the purity of the cell product was significantly reduced by pulsing the zoledronate, by using IL-15 instead of IL-2, and by combining both modifications (see FIGS. 5D-F).

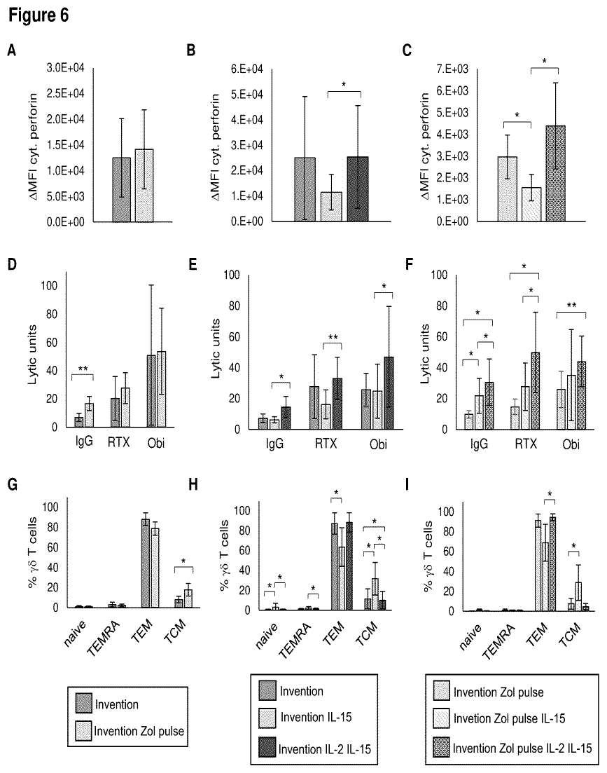

Regarding cytoplasmic perforin, the use of IL-15 instead of IL-2 led to a significant reduction both without and with zoledronate pulse (see FIGS. 6A-B). The zoledronate pulse alone and the use of IL-15 in addition to IL-2 did not lead to any significant change (see FIGS. 6A-C). The modifications had no significant effect on cytoplasmic IFN γ and CD107a expression in the degranulation assay (data not shown).

The present inventors next performed the cytotoxicity assay with the different cultivated MNC. To be able to compare the methods more clearly, the present inventors calculated the lytic units per 10⁶ effector cells.

A significant increase in lytic units could be achieved with the zoledronate pulse compared to the standard protocol when no therapeutic antibodies are added (see FIG. 6D).

The combination of IL-15 and IL-2 led to no change in lytic units both with and without antibodies, but to significant higher lytic units compared to IL-15 alone (see FIG. 6E).

Interestingly, when the modifications are combined, an increase in lytic units could be achieved without antibody when IL-15 alone or IL-15 and IL-2 were added, and with antibody when IL-15 and IL-2 were added, compared to the zoledronate pulse without modification of the interleukins (see FIG. 6F).

The present inventors divided the T cells into naive, central memory, effector memory and terminally differentiated Gamma Delta T cells based on expression of CD45RA and CD27. The stimulation with IL-15 instead of IL-2 lead to an elevated percentage of central memory Gamma Delta T cells (TCM) and to a reduced percentage of effector Gamma Delta T cells (TEM), also if combined with pulsing zoledronate (see FIGS. 6H-I).

Pulsing zoledronate with IL-2 as stimulant also resulted in more TCM (see FIG. 6G). Interestingly, combining IL-2 with IL-15 significantly reduced the percentage of TCM, but the difference was marginal (see FIG. 6H).

In summary, pulsing zoledronate and co-stimulation by the combination of IL-2 and IL-15 lead to significant changes in in vitro anti- tumor activity and the subclasses of stimulated Gamma Delta T cells.

### Time Schedule for Ex Vivo Expansion of Gamma Delta T Cells

What is the optimal time for adoptive transfer of the cells after ex vivo cultivation? Yield and purity as well as anti-tumor activity should be as high as possible. In view of all the findings, the present inventors consider the resulting cell product on day 10 to be the most suitable of the tested days for in vivo application.

### Improvement of Immunotherapeutic Cell Products by a Newly Developed Cultivation Method

The present invention comprises a GMP-compliant cultivation procedure for Gamma Delta T cells that generates a cell product with high proliferation rate, purity and anti-tumor activity. Gamma Delta T cell counts increased more than 400-fold and the cell product consisted of over 90% Gamma Delta T cells. Compared to prior art, cultivation with the present invention led to higher production of cytoplasmic perforin, to higher degranulation and to stronger cytotoxicity with and without monoclonal antibodies directed against target cells. The present invention can be rapidly implemented in the clinic and is more easily applicable for broader use in academia.

The scope of the claims should not be limited by the preferred embodiments set forth in the examples, but should be given the broadest interpretation consistent with the description as a whole.

1. A method for cultivating Gamma Delta T cells for immunotherapy, said method comprising:
(i) obtaining peripheral blood mononuclear cells (MNCs) from a donor;
(ii) optionally, isolating said MNCs by centrifugation;
(iii) culturing said MNCs in a basal medium comprising human plasma, albumin and L-Glutamine;
(iv) adding zoledronate to the medium;
(v) adding a Interleukin-2 (IL-2) to the medium; and
(vi) removing and replacing a portion of the medium with fresh medium.

2. The method of claim 1, wherein the donor is less than 40 years old.

3. The method of claim 1, wherein the zoledronate is added to the medium on day 0 of cultivating or after.

4. The method of claim 1, wherein 10 μM zoledronate is added to the medium.

5. The method of claim 1, wherein the IL-2 is added to the medium on day 2 of cultivating or after.

6. The method of claim 1, wherein 1000 U/ml IL-2 is added to the medium.

7. The method of claim 1, wherein the stop of removing and replacing a portion of the medium with fresh medium takes place on days 4, 7, 9, 11 and 14 of cultivating.

8. The method of claim 1, wherein half of the medium is removed and replaced with fresh medium.

9. The method of claim 8, wherein the fresh medium comprises 2× 1000 U/ml IL-2 for a final concentration of 1000 U/ml IL-2.

10. The method of claim 1, further comprising a step of shaking the MNCs and medium.

11. The method of claim 10, wherein the MNCs and medium are shaken at about 250 rpm.

12. The method of claim 1, wherein a mixture of IL-2 and IL-15 is added to the medium.

13. The method of claim 12, wherein the mixture of IL-2 and IL-15 is prepared by diluting IL-2 and IL-15 in PBS/5% HSA, mixing and incubating at about 37° C. and about 5% CO₂.

14. The method of claim 1, wherein a mixture of zoledronate and IL-2 is added to the medium.

15. The method of claim 14, wherein the mixture of zoledronate and IL-2 is prepared by diluting zoledronate and IL-2 in PBS/5% HSA, mixing and incubating at about 37° C. and about 5% CO₂.

16. The method of claim 1, wherein the method of cultivating is performed over 10 days.

17. Gamma Delta T cells for immunotherapy, said Gamma Delta T cells made by the method as defined in claim 1.

18. Gamma Delta T cells in combination with one or more monoclonal antibodies for immunotherapy, said Gamma Delta T cells made by the method as defined in claim 1.
